(19) United States
(12) Patent Application Publication
(10) Pub. No.: US 2010/0240546 A1
(43) Pub. Date: Sep. 23, 2010

(54) USE OF BIOMARKERS FOR THE DIAGNOSIS AND PROGNOSIS OF LUNG CANCER

(76) Inventors: Samuel Chun Lap Lo, Hong Kong (HK); Yoki Kwok Chu Butt, Hong Kong (HK); Wallace Woon Fong Leung, Hong Kong (HK)

Correspondence Address: WILKINSON & GRIST, 6TH FLOOR, PRINCE''S BUILDING, CHATER ROAD, CENTRAL, HONG KONG, HK

(21) Appl. No.: 12/408,547

(22) Filed: Mar. 20, 2009

(51) Int. Cl. C40B 30/04; C12Q 1/28; C12Q 1/37; C12Q 1/48; C12Q 1/34; G01N 33/68; C12Q 1/02; G01N 33/566; G01N 33/573; G01N 33/53

(52) U.S. Cl. 506/9; 435/28; 435/24; 435/15; 435/18; 436/86; 435/29; 436/501; 435/7.4; 435/7.92

(57) ABSTRACT

A method for identifying, diagnosing, and monitoring cancerous lung tissues in a subject may include measuring the expression of at least one biomarker in a biological sample in the subject, and comparing the expression against a normal value. When a differential expression of the at least one biomarker between the biological sample and the normal value is more than 1.5-fold, the lung tissue sample is cancerous. The at least one biomarker is selected from the group consisting of peroxiredoxin I, peroxiredoxin II, peroxiredoxin III, peroxiredoxin IV, peroxiredoxin VI, chaperonin, amyloid-P-component, annexin V, dihydropyrimidinase-like 2 protein, glutamate carboxypeptidase, 2,3-bisphosphoglycerate mutase, thymidine phosphorylase, prolyl-4 hydroxylase, selenium binding protein 1, β-mitochondrial ATP synthase H⁺ transporting F1 complex, laminin-binding protein, minichromosome maintenance deficient protein 5 variant, keratin 9, keratin 10, napsin A aspartic peptidase, M2-type pyruvate kinase, and apolipoprotein A-I.

### BACKGROUND

Lung cancer is the leading cause of cancer deaths worldwide, and non-small cell lung carcinoma (NSCLC) accounts for nearly 85% of all cases of lung cancers. The progression of cancer is usually manifested as uncontrolled growth in cancerous tissues, construction of new blood vessels and invasion into adjacent tissues. These manifestations may bring about concomitant changes in expression of proteins, including their formation, concentrations and interactions with other molecules in the cancerous tissues.

Occurrence of NSCLC is usually diagnosed at a late stage, resulting in high mortality. While early detection of NSCLC is a crucial step to decrease the high mortality rate, there are currently no reliable methods for early detection.

Current diagnostic tests for lung cancer may include chest X-ray, sputum cytology, bronchoscopy, image scans and biopsy. However, these tests can only detect the occurrence of lung cancer at a relatively late stage. Furthermore, because of the limitations imposed on equipment and the establishments required, these methods are not suitable for large-scale diagnostic screening.

Consequently, there is an urgent need to develop an efficient method to detect early stages of NSCLC for screening, diagnostic and prognostic purposes. It would also be desirable if the method could allow further determination of the efficacy of treatment of lung cancer by surgery, radiation and chemotherapy, as well as further the design of targeted drugs for cancer treatment.

### BRIEF SUMMARY

According to one aspect, a method for identifying, diagnosing, and monitoring cancerous lung tissues in a subject may include measuring the expression of at least one biomarker in a biological sample in the subject, and comparing the expression against a normal value. When a differential expression of the at least one biomarker between the biological sample and the normal value is more than 1.5-fold, the lung tissue sample is cancerous. The at least one biomarker is selected from the group consisting of peroxiredoxin I, peroxiredoxin II, peroxiredoxin III, peroxiredoxin IV, peroxiredoxin VI, chaperonin, amyloid-P-component, annexin V, dihydropyrimidinase-like 2 protein, glutamate carboxypeptidase, 2,3-bisphosphoglycerate mutase, thymidine phosphorylase, prolyl-4 hydroxylase, selenium binding protein 1, β-mitochondrial ATP synthase H⁺ transporting F1 complex, laminin-binding protein, minichromosome maintenance deficient protein 5 variant, keratin 9, keratin 10, napsin A aspartic peptidase, M2-type pyruvate kinase, and apolipoprotein A-I.

According to another aspect, a method of determining the effectiveness of a treatment diagnosed with cancerous lung tissues may include measuring the expression of at least one biomarker in a first biological sample from the patient before treatment, measuring the expression of the at least one biomarker in a second biological sample from the same patient taken during the treatment, comparing the expression of the first sample against a normal value, and comparing the expression of the second sample after treatment against the normal value. When a differential expression of the at least one biomarker between the first sample and the normal value is more than 1.5-fold than a differential expression of the at least one biomarker between the second sample and the normal value, the treatment is effective.

### BRIEF DESCRIPTION OF THE DRAWINGS

FIG. 1A shows the location of various biomarkers in a 2-DE proteome map of protein extracts from the normal lung tissue of a first patient.

FIG. 1B shows the location of various biomarkers in a 2-DE proteome map of protein extracts from the cancerous lung tissue of a first patient.

FIG. 2A depicts the enlarged gel regions showing peroxiredoxin II expressed in non-tumor and tumor tissues of a first patient sample.

FIG. 2B depicts the enlarged gel regions showing peroxiredoxin II expressed in non-tumor and tumor tissues of a second patient sample.

FIG. 2C depicts the enlarged gel regions showing peroxiredoxin II expressed in non-tumor and tumor tissues of a third patient sample.

FIG. 3A depicts the enlarged gel regions showing peroxiredoxin II expressed in non-tumor and tumor tissues of a first patient sample.

FIG. 3B depicts the enlarged gel regions showing peroxiredoxin II expressed in non-tumor and tumor tissues of a second patient sample.

FIG. 3C depicts the enlarged gel regions showing peroxiredoxin II expressed in non-tumor and tumor tissues of a third patient sample.

FIG. 4A depicts the enlarged gel regions showing chaperonin expressed in non-tumor and tumor tissues of a first patient sample.

FIG. 4B depicts the enlarged gel regions showing chaperonin expressed in non-tumor and tumor tissues of a second patient sample.

FIG. 4C depicts the enlarged gel regions showing chaperonin expressed in non-tumor and tumor tissues of a third patient sample.

FIG. 5A depicts the enlarged gel regions showing amyloid-P-component expressed in non-tumor and tumor tissues of a first patient sample.

FIG. 5B depicts the enlarged gel regions showing amyloid-P-component expressed in non-tumor and tumor tissues of a second patient sample.

FIG. 5C depicts the enlarged gel regions showing amyloid-P-component expressed in non-tumor and tumor tissues of a third patient sample.

FIG. 6A depicts the enlarged gel regions showing annexin V expressed in non-tumor and tumor tissues of a first patient sample.

FIG. 6B depicts the enlarged gel regions showing annexin V expressed in non-tumor and tumor tissues of a second patient sample.

FIG. 6C depicts the enlarged gel regions showing annexin V in non-tumor and tumor tissues of a third patient sample.

FIG. 7A depicts the enlarged gel regions showing dihydropyrimidinase-like 2 protein expressed in non-tumor and tumor tissues of a first patient sample.

FIG. 7B depicts the enlarged gel regions showing dihydropyrimidinase-like 2 protein expressed in non-tumor and tumor tissues of a second patient sample.

FIG. 7C depicts the enlarged gel regions showing dihydropyrimidinase-like 2 protein expressed in non-tumor and tumor tissues of a third patient sample.

FIG. 8A depicts the enlarged gel regions showing glutamate carboxypeptidase expressed in non-tumor and tumor tissues of a first patient sample.

FIG. 8B depicts the enlarged gel regions showing glutamate carboxypeptidase expressed in non-tumor and tumor tissues of a second patient sample.

FIG. 8C depicts the enlarged gel regions showing glutamate carboxypeptidase expressed in non-tumor and tumor tissues of a third patient sample.

FIG. 9A depicts the enlarged gel regions showing 2,3-bisphosphoglycerate mutase expressed in non-tumor and tumor tissues of a first patient sample.

FIG. 9B depicts the enlarged gel regions showing 2,3-bisphosphoglycerate mutase expressed in non-tumor and tumor tissues of a second patient sample.

FIG. 9C depicts the enlarged gel regions showing 2,3-bisphosphoglycerate mutase expressed in non-tumor and tumor tissues of a third patient sample.

FIG. 10A depicts the enlarged gel regions showing thymidine phosphorylase expressed in non-tumor and tumor tissues of a first patient sample.

FIG. 10B depicts the enlarged gel regions showing thymidine phosphorylase expressed in non-tumor and tumor tissues of a second patient sample.

FIG. 10C depicts the enlarged gel regions showing thymidine phosphorylase expressed in non-tumor and tumor tissues of a third patient sample.

FIG. 11A depicts the enlarged gel regions showing prolyl-4 hydroxylase expressed in non-tumor and tumor tissues of a first patient sample.

FIG. 11B depicts the enlarged gel regions showing prolyl-4 hydroxylase expressed in non-tumor and tumor tissues of a second patient sample.

FIG. 11C depicts the enlarged gel regions showing prolyl-4 hydroxylase expressed in non-tumor and tumor tissues of a third patient sample.

FIG. 12A depicts the enlarged gel regions showing selenium binding protein 1 expressed in non-tumor and tumor tissues of a first patient sample.

FIG. 12B depicts the enlarged gel regions showing selenium binding protein 1 expressed in non-tumor and tumor tissues of a second patient sample.

FIG. 12C depicts the enlarged gel regions showing selenium binding protein 1 expressed in non-tumor and tumor tissues of a third patient sample.

FIG. 13A depicts the enlarged gel regions showing β-mitochondrial ATP synthase H⁺ transporting F1 complex expressed in non-tumor and tumor tissues of a first patient sample.

FIG. 13B depicts the enlarged gel regions showing β-mitochondrial ATP synthase H⁺ transporting F1 complex expressed in non-tumor and tumor tissues of a second patient sample.

FIG. 13C depicts the enlarged gel regions showing β-mitochondrial ATP synthase H⁺ transporting F1 complex expressed in non-tumor and tumor tissues of a third patient sample.

FIG. 14A depicts the enlarged gel regions showing laminin-binding protein expressed in non-tumor and tumor tissues of a first patient sample.

FIG. 14B depicts the enlarged gel regions showing laminin-binding protein expressed in non-tumor and tumor tissues of a second patient sample.

FIG. 14C depicts the enlarged gel regions showing laminin-binding protein expressed in non-tumor and tumor tissues of a third patient sample.

FIG. 15A depicts the enlarged gel regions showing minichromosome maintenance deficient protein 5 variant expressed in non-tumor and tumor tissues of a first patient sample.

FIG. 15B depicts the enlarged gel regions showing minichromosome maintenance deficient protein 5 variant expressed in non-tumor and tumor tissues of a second patient sample.

FIG. 15C depicts the enlarged gel regions showing minichromosome maintenance deficient protein 5 variant expressed in non-tumor and tumor tissues of a third patient sample.

FIG. 16A depicts the enlarged gel regions showing keratin 9 expressed in non-tumor and tumor tissues of a first patient sample.

FIG. 16B depicts the enlarged gel regions showing keratin 9 expressed in non-tumor and tumor tissues of a second patient sample.

FIG. 16C depicts the enlarged gel regions showing keratin 9 expressed in non-tumor and tumor tissues of a third patient sample.

FIG. 17A depicts the enlarged gel regions showing keratin 10 expressed in non-tumor and tumor tissues of a first patient sample.

FIG. 17B depicts the enlarged gel regions showing keratin 10 expressed in non-tumor and tumor tissues of a second patient sample.

FIG. 17C depicts the enlarged gel regions showing keratin 10 expressed in non-tumor and tumor tissues of a third patient sample.

FIG. 18A depicts the enlarged gel regions showing napsin A aspartic peptidase expressed in non-tumor and tumor tissues of a first patient sample.

FIG. 18B depicts the enlarged gel regions showing napsin A aspartic peptidase expressed in non-tumor and tumor tissues of a second patient sample.

FIG. 18C depicts the enlarged gel regions showing napsin A aspartic peptidase expressed in non-tumor and tumor tissues of a third patient sample.

FIG. 19A depicts the enlarged gel regions showing M2-type pyruvate kinase expressed in non-tumor and tumor tissues of a first patient sample.

FIG. 19B depicts the enlarged gel regions showing M2-type pyruvate kinase expressed in non-tumor and tumor tissues of a second patient sample.

FIG. 19C depicts the enlarged gel regions showing M2-type pyruvate kinase expressed in non-tumor and tumor tissues of a third patient sample.

FIG. 20A depicts the enlarged gel regions showing apolipoprotein A-I expressed in non-tumor and tumor tissues of a first patient sample.

FIG. 20B depicts the enlarged gel regions showing apolipoprotein A-I expressed in non-tumor and tumor tissues of a second patient sample.

FIG. 20C depicts the enlarged gel regions showing apolipoprotein A-I expressed in non-tumor and tumor tissues of a third patient sample.

FIG. 21A is a figure illustrating the western blot analysis of serum amyloid P component in tumor tissues and the adjacent non-tumor tissues from 10 NSCLC patients.

FIG. 21B depicts the fold change in the expression level of amyloid P component in lung tumor tissues, compared with the adjacent non-tumor lung tissues.

FIG. 22A is a figure illustrating the western blot analysis of chaperonin in tumor tissues and the adjacent non-tumor tissues from 10 NSCLC patients.

FIG. 22B depicts the fold change in the expression level of chaperonin in lung tumor tissues, compared with the adjacent non-tumor lung tissues.

FIG. 23A is a figure illustrating the western blot analysis of peroxiredoxin II in tumor tissues and the adjacent non-tumor tissues from 10 NSCLC patients.

FIG. 23B depicts the fold change in the expression level of peroxiredoxin II in lung tumor tissues, compared with the adjacent non-tumor lung tissues.

FIG. 24A is a figure illustrating the western blot analysis of peroxiredoxin III in tumor tissues and the adjacent non-tumor tissues from 10 NSCLC patients.

FIG. 24B depicts the fold change in the expression level of peroxiredoxin III in lung tumor tissues, compared with the adjacent non-tumor lung tissues.

FIG. 25A is a figure illustrating the western blot analysis of peroxiredoxin VI in tumor tissues and the adjacent non-tumor tissues from 10 NSCLC patients.

FIG. 25B depicts the fold change in the expression level of peroxiredoxin VI in lung tumor tissues, compared with the adjacent non-tumor lung tissues.

FIG. 26 is a dot chart showing the log value of fold change of the expression level of amyloid P component, chaperonin, peroxiredoxin II, III and VI in tumor tissues from 10 NSCLC patients.

FIG. 27 is a bar chart showing the fold change in the expression levels of annexin V, amyloid P component (APC), chaperonin (HSP60), peroxiredoxin (Prx)-II, Prx-III and Prx-VI in cancerous lung tissues from NSCLC patient.

### DETAILED DESCRIPTION

Reference will now be made in detail to a particular embodiment of the invention, examples of which are also provided in the following description. Exemplary embodiments of the invention are described in detail, although it will be apparent to those skilled in the relevant art that some features that are not particularly important to an understanding of the invention may not be shown for the sake of clarity.

Furthermore, it should be understood that the invention is not limited to the precise embodiments described below, and that various changes and modifications thereof may be effected by one skilled in the art without departing from the spirit or scope of the invention. For example, elements and/or features of different illustrative embodiments may be combined with each other and/or substituted for each other within the scope of this disclosure and appended claims. In addition, improvements and modifications which may become apparent to persons of ordinary skill in the art after reading this disclosure, the drawings, and the appended claims are deemed within the spirit and scope of the present invention.

A method for identifying, diagnosing and monitoring cancerous lung tissue in a subject may include measuring the expression of at least one biomarker in a biological sample in the subject, and comparing the expression against a normal value. When a differential expression of the at least one biomarker between the biological sample and the normal value is more than 1.5-fold, the lung tissue sample is cancerous.

The term “biological sample” means lung tissue, lung tissue biopsy and/or serum obtained from subjects suspected to have lung cancer or patients suffered from lung cancer. The biological sample may include lung tissue, lung tissue biopsy and/or serum.

The term “differential expression” means the change in the abundance of the biomarker in one sample as compared against the normal value. Preferably, the said at least one biomarker is at least two biomarkers, selected from those in Table 1. The term “expression” includes the expression of the whole protein species, the expression of the fragments of such proteins, the expression of mRNA encoding such proteins, and/or antibodies specifically against such proteins.

The term “normal value” means a range of a measurement or a ratio in a normal sample, a range of standardized value obtained from a group of healthy subjects.

A method of determining the effectiveness of a treatment diagnosed with cancerous lung tissues may include measuring the expression of at least one biomarker in a first biological sample from the patient before treatment, measuring the expression of the at least one biomarker in a second biological sample from the same patient taken during the treatment, comparing the expression of the first sample against a normal value, and comparing the expression of the second sample after treatment against the normal value. When a differential expression of the at least one biomarker between the first sample and the normal value is more than 1.5-fold than a differential expression of the at least one biomarker between the second sample and the normal value, the treatment is effective.

1. Biomarkers

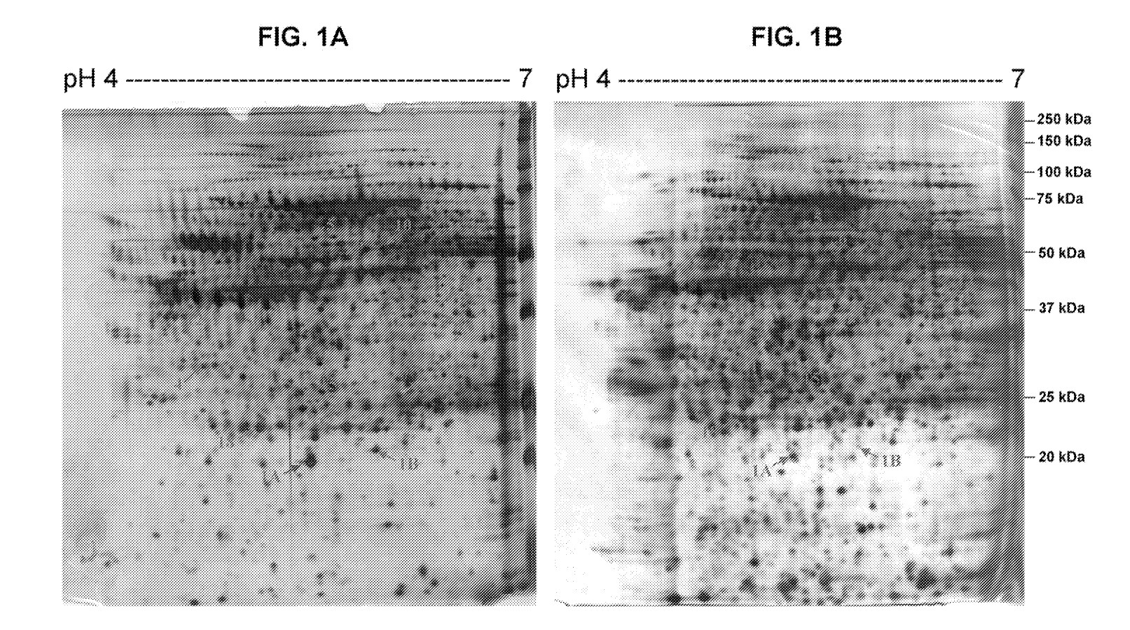

The biomarkers may include peroxiredoxin (Prx)-I, Prx-II, Prx-III, Prx-IV, Prx-VI, chaperonin, amyloid-P-component (APC), annexin V, dihydropyrimidinase-like 2 protein, glutamate carboxypeptidase, 2,3-bisphosphoglycerate mutase, thymidine phosphorylase, prolyl-4 hydroxylase, selenium binding protein 1, β-mitochondrial ATP synthase H⁺ transporting F1 complex, laminin-binding protein, minichromosome maintenance deficient protein 5 variant, keratin 9, keratin 10, napsin A aspartic peptidase, M2-type pyruvate kinase, and apolipoprotein A-I. The locations of the biomarkers expressed in non-tumor and tumor tissues in a 2-DE proteome map are depicted in FIGS. 1A and 1B, respectively.

1.1 Peroxiredoxin

The biomarkers may include peroxiredoxin (Prx), which has a down-regulated expression in lung tumor tissues. The peroxiredoxin may include peroxiredoxin I (Prx-I), peroxiredoxin II (Prx-II), peroxiredoxin III (Prx-III), peroxiredoxin IV (Prx-IV), and peroxiredoxin VI (Prx-VI).

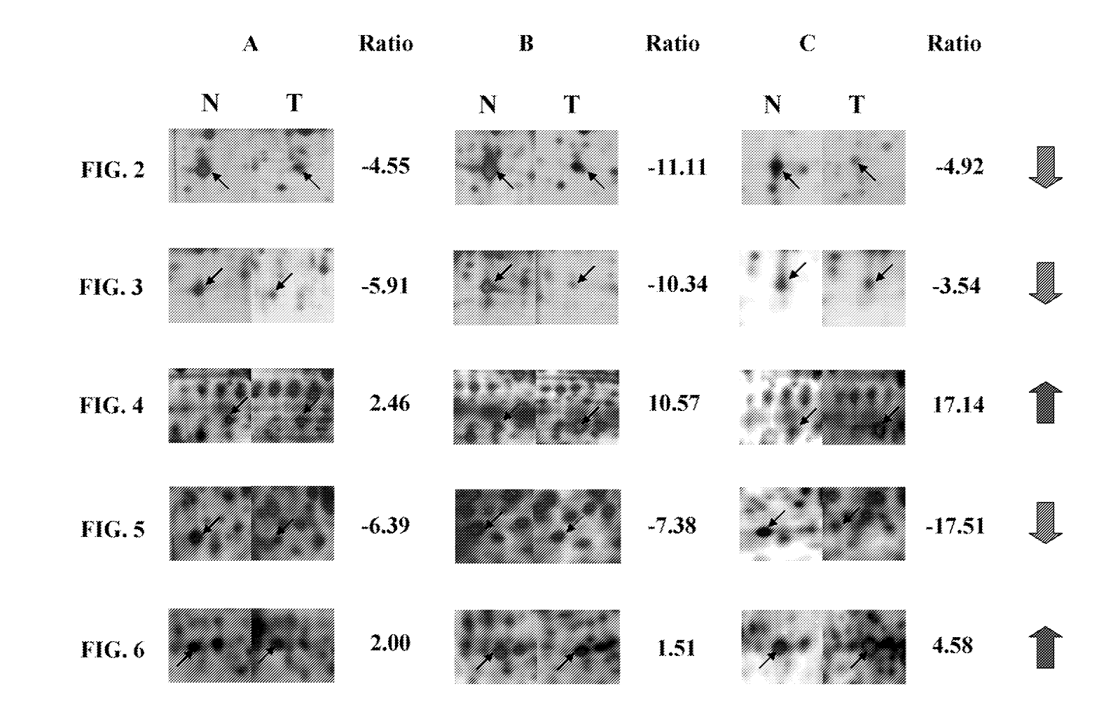

Peroxiredoxin I may also be known as thioredoxin peroxidase 2, thioredoxin-dependent peroxide reductase 2, and proliferation-associated gene protein. Peroxiredoxin I has an accession number of Q06830, and a molecular weight of 22.1 kDa. Peroxiredoxin II may also be known as thioredoxin peroxidase 1, thioredoxin-dependent peroxide reductase 1, and thiol-specific antioxidant protein. Peroxiredoxin II has an accession number of P32119, and a molecular weight of 21.7 kDa. The enlarged gel regions showing peroxiredoxin II expressed in non-tumor and tumor tissues are depicted in FIGS. 2A to 2C and FIGS. 3A to 3C. Peroxiredoxin III may also be known as antioxidant protein 1. Peroxiredoxin III has an accession number of P30048, and a molecular weight of 27.7 kDa. Peroxiredoxin IV may also be known as thioredoxin peroxidase A0372, thioredoxin-dependent peroxide reductase A0372, and antioxidant enzyme AOE372. Peroxiredoxin IV has an accession number of Q13162, and a molecular weight of 30.5 kDa. Peroxiredoxin VI may also be known as antioxidant protein 2, 1-Cys peroxiredoxins, acidic calcium-independent phospholipase A2, and non-selenium glutathione peroxidase. Peroxiredoxin VI has an accession number of P30041, and a molecular weight of 25.0 kDa.

Prx is an antioxidant enzyme detoxifying reactive oxygen species. Prx may reduce hydrogen peroxide (H₂O₂) and alkyl hydroperoxide to water (H₂O) and alcohol through a catalytic system that uses thioredoxin (Trx), Trx reductase (TR) and NADPH. Prx I and Prx II are cytoplasm. Prx III is only found in mitochondria. Prx IV is produced in the endoplasmic reticulum. Prx V is found either in lysosomes or peroxisomes.

1.2 Chaperonin

The biomarkers may also include chaperonin, which has an up-regulated expression in lung tumor tissues. Chaperonin may also be known as chaperonin 60, heat shock protein 60, mitochondrial matrix protein P1, and P60 lymphocyte protein. Chaperonin has an accession number of P10809, and a molecular weight of 61.0 kDa. The enlarged gel regions showing chaperonin expressed in non-tumor and tumor tissues are depicted in FIGS. 4A to 4C.

Chaperonin or heat shock protein 60 is a highly conserved protein mostly localized in the mitochondrial matrix. Chaperonin may play a role in the regulation of a variety of cellular functions. The primary function is to assist in mitochondrial protein folding, unfolding and degradation. Besides, chaperonin may also be associated with the regulation of protein turnover and the cellular redox state.

1.3 Amyloid P Component

The biomarkers may also include amyloid P component (APC), which has a down-regulated expression in lung tumor tissues. APC may also be known as 9.5S alpha-1-glycoprotein. APC has an accession number of P02743, and a molecular weight of 25.4 kDa. APC is commonly found in serum, and is known as a prototypic acute phase reactant and the protein that binds to dying cells facilitating their swift removal by phagocytes. The enlarged gel regions showing APC expressed in non-tumor and tumor tissues are depicted in FIGS. 5A to 5C.

1.4 Annexin V

The biomarkers may also include annexin V, which has an up-regulated expression in lung tumor tissues. Annexin V may also be known as annexin A5, lipocortin V, endonexin II, calphobinlin I, placental anticoagulant protein I, placental anticoagulant protein 4, thromboplastin inhibitor, vascular anticoagulant-alpha, anchorin CII. Annexin V has an accession number of P08758, and a molecular weight of 35.8 kDa. The enlarged gel regions showing annexin V expressed in non-tumor and tumor tissues are depicted in FIGS. 6A to 6C.

Annexins are characterized as a structurally related family of calcium-binding proteins that have a domain that binds to negatively charged phospholipids in a calcium dependent manner and contain an annexin repeat with 70 amino acids. In humans, the annexins are found inside the cell, whereas some annexins (annexin I, II and V) may also be found outside the cellular environment, such as in the blood. Annexins are involved in various cellular processes, such as the regulation of calcium ion currents, membrane trafficking, cellular adhesion, cellular signaling, the maintenance of cytoskeleton and extracellular matrix integrity, development and differentiation, inflammation and blood coagulation.

1.5 Dihydropyrimidinase-Like 2 Protein

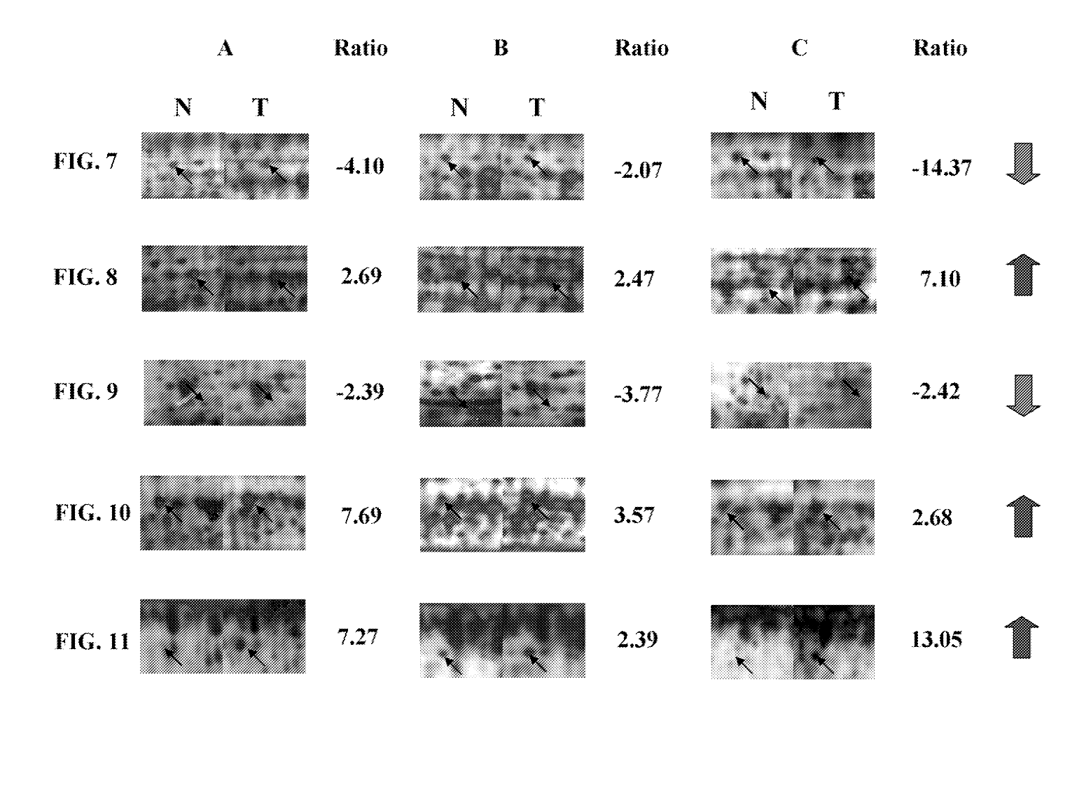

The biomarkers may also include dihydropyrimidinase-like 2 protein, which has a down-regulated expression in lung tumor tissues. Dihydropyrimidinase-like 2 protein may also be known as dihydropyrimidinase-like 2 variant, and collapsin response mediator protein-2. Dihydropyrimidinase-like 2 protein has an accession number of Q16555, and a molecular weight of 62.3 kDa. The enlarged gel regions showing dihydropyrimidinase-like 2 protein expressed in non-tumor and tumor tissues are depicted in FIGS. 7A to 7C.

Dihydropyrimidinase-like 2 protein is a member of the dihydropyrimidinase-related family proteins (Type 1-5) exclusively expressed in the nervous system. In the nervous system, these proteins are mainly involved in axonal outgrowth and path-finding through the transmission and modulation of extracellular signals.

1.6 Glutamate Carboxypeptidase

The biomarkers may also include glutamate carboxypeptidase, which has an up-regulated expression in lung tumor tissues. Glutamate carboxypeptidase may also be known as membrane glutamate carboxypeptidase, N-acetylated-alpha-linked acidic dipeptidase I, pteroylpoly-gamma-glutamate carboxypeptidase, folylpoly-gamma-glumate carboxypeptidase, folate hydrolase 1, and prostate-specific membrane antigen. Glutamate carboxypeptidase has an accession number of Q04609, and a molecular weight of 84.3 kDa. The enlarged gel regions showing glutamate carboxypeptidase expressed in non-tumor and tumor tissues are depicted in FIGS. 8A to 8C.

Glutamate carboxypeptidase is a transmembrane glycoprotein with a short intracellular, NH₂-terminal domain and a large extracellular COOH-terminal domain. Its structure is identical to prostate-specific membrane antigen (PSMA) and folate hydrolase 1. Glutamate carboxypeptidase may have both folate hydrolase and N-acetylated-alpha-liked-acidic dipeptidase activity, and may also exhibit a dipeptidyl-peptidase IV type activity. In the intestine, glutamate carboxypeptidase may be required for the uptake of folate. In the brain, glutamate carboxypeptidase may modulate excitatory neurotransmission through the hydrolysis of the neuropeptide, N-aceylaspartylglutamate, thereby releasing glutamate.

1.7 2,3-bisphosphoglycerate Mutase

The biomarkers may also include 2,3-bisphosphoglycerate mutase, which has a down-regulated expression in lung tumor tissues. 2,3-bisphosphoglycerate mutase may also be known as 2,3-bisphosphoglycerate synthase and 2,3-bisphophoglycerate synthase-phosphatase. 2,3-bisphosphoglycerate mutase has an accession number of P07738, and a molecular weight of 30.0 kDa. The enlarged gel regions showing 2,3-bisphosphoglycerate mutase expressed in non-tumor and tumor tissues are depicted in FIGS. 9A to 9C.

2,3-bisphosphoglycerate mutase (BPGM) catalyzes the conversion of 1,3-bisphosphoglycerate (1,3-BPG) to 2,3-bisphosphoglycerate (2,3-BPG). In mammalian tissues, BPGM may have 2-phosphoglycolate-stimulated 2,3-bisphosphoglycerate phosphatase (BPGP) activity. The final product of BPGM, 2,3-BPG, may play an important role in erythrocytes, in which it acts as an allosteric inhibitor of hemoglobin. The binding of 2,3-BPG decreases hemoglobin's affinity for oxygen and facilitates the transfer of oxygen to tissues. Excess amounts of 2,3-BPG re-enter the glycolytic pathway through a phosphatase catalyzed conversion to glycerate-3-phosphate.

1.8 Thymidine Phosphorylase

The biomarkers may also include thymidine phosphorylase (TP), which has an up-regulated expression in lung tumor tissues. Thymidine phosphorylase may also be known as pyrimidine phosphorylase, thymidine-orthophosphate deoxyribosyltransferase, animal growth regulators, blood platelet-derived endothelial cell, growth factors, blood platelet-derived endothelial cell growth factor, deoxythymidine phosphorylase, gliostatins, pyrimidine deoxynucleoside phosphorylase, and thymidine:phosphate deoxy-D-ribosyltransferase. Thymidine phosphorylase has an accession number of P19971, and a molecular weight of 49.9 kDa. The enlarged gel regions showing thymidine phosphorylase expressed in non-tumor and tumor tissues are depicted in FIGS. 10A to 10C.

In the presence of inorganic orthophosphate, thymidine phosphorylase may catalyze the reversible phosphorolysis of thymidine and 2-deoxyuridine to their corresponding bases, thymine and 2-deoxyribose-1-phosphate. These produced molecules may then be utilized as carbon and energy sources or in the rescue of pyrimidine bases for nucleotide synthesis. Besides, thymidine phosphorylase may have both angiogenic and chemotactic properties.

1.9 Prolyl-4-hydroxylase

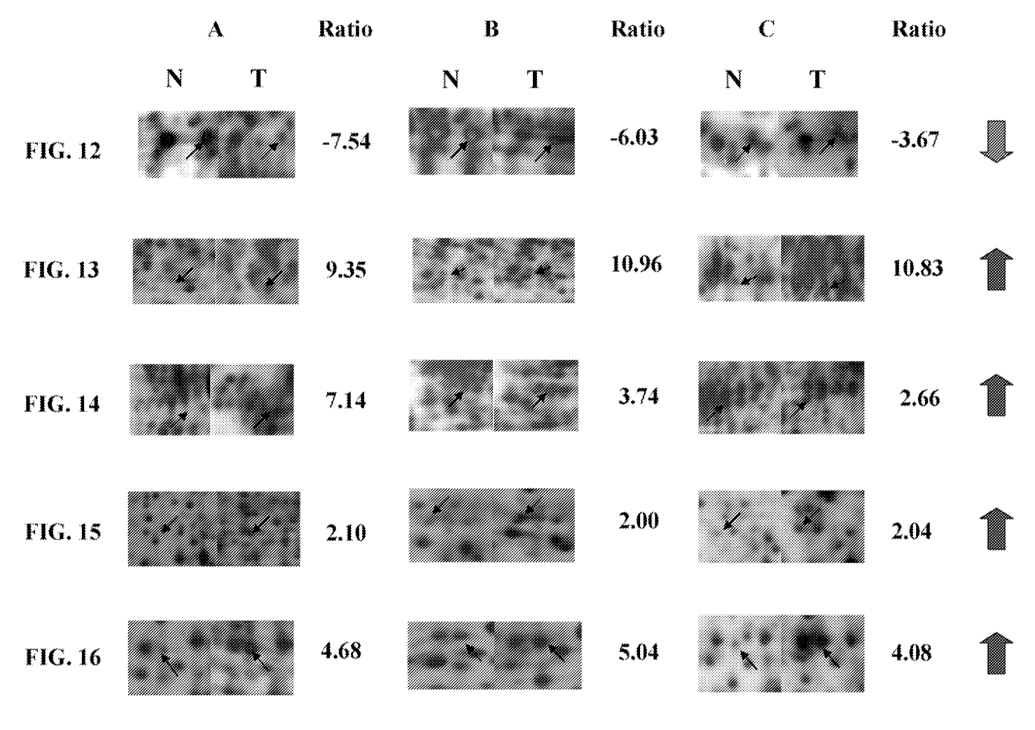

The biomarkers may also include prolyl-4-hydroxylase, which has an up-regulated expression in lung tumor tissues. Prolyl-4-hydroxylase may also be known as protein disulfide isomerase, cellular thyroid hormone-binding protein, procollagen proline, 2-oxyglutarate 4-dioxygenase, proline-4-hydroxylase. Prolyl-4-hydroxylase has an accession number of P07237, and a molecular weight of 57.1 kDa. The enlarged gel regions showing prolyl-4-hydroxylase expressed in non-tumor and tumor tissues are depicted in FIGS. 11A to 11C.

Prolyl-4-hydroxylase is a key post-translational modifying enzyme in collagen synthesis, and it also participates in antioxidation and detoxification reactions.

1.10 Selenium Binding Protein 1

The biomarkers may also include selenium binding protein 1, which has a down-regulated expression in lung tumor tissues. Selenium binding protein 1 may also be known as 56 kDa selenium-binding protein. Selenium binding protein 1 has an accession number of Q13228, and a molecular weight of 52.4 kDa. The enlarged gel regions showing selenium binding protein 1 expressed in non-tumor and tumor tissues are depicted in FIGS. 12A to 12C.

Selenium is an essential micronutrient that exhibits potent anticarcinogenic properties, and deficiency of selenium may cause carcinomas and certain neurologic diseases.

1.11 β-mitochondrial ATP Synthase, H⁺ Transporting F1 Complex

The biomarkers may also include β-mitochondrial ATP synthase, H⁺ transporting F1 complex, which has an up-regulated expression in lung tumor tissues. β-mitochondrial ATP synthase, H⁺ transporting F1 complex may also be known as ATP synthase subunit beta, mitochondrial. mitochondrial ATP synthase, H⁺ transporting F1 complex has an accession number of P06579, and a molecular weight of 56.5 kDa. The enlarged gel regions showing mitochondrial ATP synthase, H⁺ transporting F1 complex expressed in non-tumor and tumor tissues are depicted in FIGS. 13A to 13C. H⁺-ATP synthase is the inner mitochondrial protein complex responsible for the synthesis of most cellular ATP requirements. The activity and molecular components of the H⁺-ATP synthase may be essential for efficient execution of programmed cell death.

1.12 Laminin-Binding Protein

The biomarkers may also include laminin-binding protein, which has an up-regulated expression in lung tumor tissues. Laminin-binding protein may also be known as 40S ribosomal protein SA, laminin receptor 1, 34/67 kDa laminin receptor, colon carcinoma laminin-binding protein, multidrug resistance-associated protein MGr1-Ag. Laminin-binding protein has an accession number of P08865, and a molecular weight of 67.0 kDa. The enlarged gel regions showing laminin-binding protein expressed in non-tumor and tumor tissues are depicted in FIGS. 14A to 14C.

Laminin binding protein or laminin receptor is a non-integrin protein, which is co-expressed and co-regulated with the α6 integrin subunit, and is physically associated with this integrin subunit on the cell membrane. Laminin binding protein may be involved in regulating and stabilizing the interaction of Laminin with the α6 integrin.

1.13 Minichromosome Maintenance Deficient Protein 5 Variant

The biomarkers may also include minichromosome maintenance deficient protein 5 variant, which has an up-regulated expression in lung tumor tissues. Minichromosome maintenance deficient protein 5 variant may also be known as minichromosome maintenance complex component 5 DNA replication licensing factor, minichromosome maintenance deficient 5 (cell division cycle 46), MCM5 minichromosome maintenance deficient 5, cell division cycle 46. Mitochromosome maintenance deficient protein 5 variant has an accession number of Q53FG5, and a molecular weight of 82.2 kDa. The enlarged gel regions showing minichromosome maintenance deficient protein 5 variant expressed in non-tumor and tumor tissues are depicted in FIGS. 15A to 15C.

Minichromosome maintenance (MCM) protein family consists of six members (MCM2-7) and is a conserved group of DNA-binding proteins. MCM proteins may be involved in the regulation of DNA synthesis, in which they may be specifically up-regulated in the transition from the G0 to G1/S phase of the cell cycle.

1.14 Keratin

The biomarkers may also include keratin. Keratin may include keratin 9 and keratin 10, both of which have an up-regulated expression in lung tumor tissues. Keratin 9 may also be known as cytokeratin 9. Keratin 9 has an accession number of P35527, and a molecular weight of 61.2 kDa. The enlarged gel regions showing keratin 9 expressed in non-tumor and tumor tissues are depicted in FIGS. 16A to 16C.

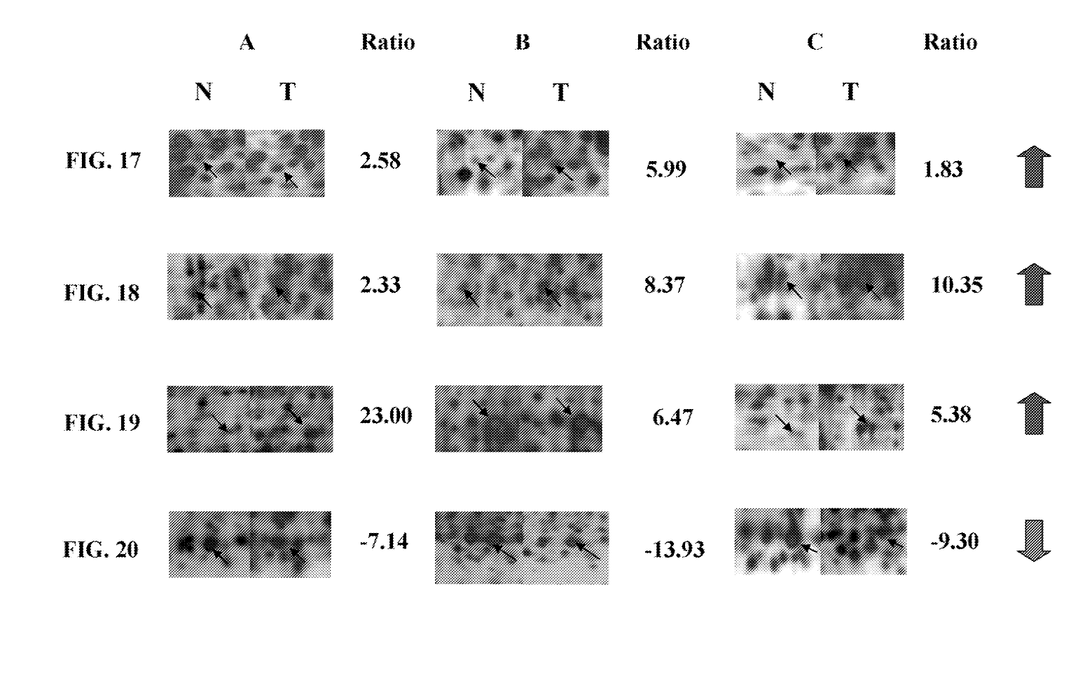

Keratin 10 may also be known as cytokeratin 10. Keratin 10 has an accession number of P13645, and a molecular weight of 59.5 kDa. The enlarged gel regions showing keratin 10 expressed in non-tumor and tumor tissues are depicted in FIGS. 17A to 17C.

Keratins are fibrous structural proteins with molecular weight ranging from 44 to 66 kDa. There are nearly 54 keratin gene members in human, including 28 type I keratin genes (K9-K10, K12-K28 and K31-K40) and 26 type II keratin genes (K1-K8 and K71-K86). Out of these human keratins, nearly 50% are specifically expressed in the hair follicle. In addition to functioning as intracellular skeletal structures, keratins are important for the mechanical stability and integrity of epithelial cells and tissues, and may participates in regulatory mechanisms and intracellular signaling pathways.

1.15 Napsin A Aspartic Peptidase

The biomarkers may also include napsin A aspartic peptidase, which has an up-regulated expression in lung tumor tissues. Napsin A aspartic peptidase may also be known as napsin-1, aspartyl protease 4. Napsin A aspartic peptidase has an accession number of Q8WWD9, and a molecular weight of 45.4 kDa. The enlarged gel regions showing napsin A aspartic peptidase expressed in non-tumor and tumor tissues are depicted in FIGS. 18A to 18C.

Napsin A aspartic peptidase is a member of the aspartic protease family, which is expressed predominantly in the cytoplasm of cells of lung (type II pneumocytes) and kidney.

1.16 M2-Type Pyruvate Kinase

The biomarkers may also include M2-type pyruvate kinase, which has an up-regulated expression in lung tumor tissues. M2-type pyruvate kinase may also be known as pyruvate kinase isozymes M1/M2, pyruvate muscle isozyme, pyruvate kinas 2/3, cytosolic thyroid hormone-binding protein. M2-type pyruvate kinase has an accession number of P14618, and a molecular weight of 57.9 kDa. The enlarged gel regions showing M2-type pyruvate kinase expressed in non-tumor and tumor tissues are depicted in FIGS. 19A to 19C.

Pyruvate kinase is a glycolytic enzyme that may catalyze the transfer of a phosphoryl group from phosphoenolpyruvate (PEP) to ADP, generating ATP. There are 4 isozymes of pyruvate kinase in mammals: L, R, M1 and M2. Pyruvate kinases are tissue-specific. L type is the major isozyme in the liver and kidney. R is found in red cells. M1 is the main form in muscle, heart and brain. M2 is found in early fetal tissues as well as in most cancer cells. The L, R and M1 pyruvate kinase may be consistently altered and replaced by M2 pyruvate kinase during the neoplastic process.

1.17 Apolipoprotein A-I

The biomarkers may also include apolipoprotein A-I, which has a down-regulated expression in lung tumor tissues. Apolipoprotein A-I has an accession number of P02647, and a molecular weight of 30.8 kDa. Apolipoprotein A-I is the major protein component of high-density lipoprotein (HDL) in the plasma, which is synthesized in the liver and small intestine and secreted in blood. The enlarged gel regions showing apolipoprotein A-I expressed in non-tumor and tumor tissues are depicted in FIGS. 20A to 20C.

A list of the biomarkers is summarized in Table 1 below.

TABLE 1
Summary of biomarkers

| | | | | Theoretical |
|---|---|---|---|---|
| | | Expression in | | Molecular |
| Name | | Lung Tissues | Accession | Weight |
| 1. | Peroxiredoxin I | Down-regulated | Q06830 | 22.1 kDa |
| 2. | Peroxiredoxin II | Down-regulated | P32119 | 21.9 kDa |
| 3. | Peroxiredoxin III | Down-regulated | P30048 | 27.7 kDa |
| 4. | Peroxiredoxin IV | Down-regulated | Q13162 | 30.5 kDa |
| 5. | Peroxiredoxin VI | Down-regulated | P30041 | 25.0 kDa |
| 6. | Chaperonin | Up-regulated | P10809 | 61.0 kDa |
| 7. | Amyloid P component | Down-regulated | P02743 | 25.4 kDa |
| 8. | Annexin V | Up-regulated | P08758 | 35.9 kDa |
| 9. | Dihydropyrimidinase-like 2 protein | Down-regulated | Q16555 | 62.3 kDa |
| 10. | Glutamate carboxypeptidase | Up-regulated | Q04609 | 84.3 kDa |
| 11. | 2,3-bisphosphoglycerate mutase | Down-regulated | P07738 | 30.0 kDa |
| 12. | Thymidine phosphorylase | Up-regulated | P19971 | 49.9 kDa |
| 13. | Prolyl-4 hydroxylase | Up-regulated | P07237 | 57.1 kDa |
| 14. | Selenium binding protein 1 | Down-regulated | Q13228 | 52.4 kDa |
| 15. | β-mitochondrial ATP synthase H⁺ | Up-regulated | P06579 | 56.6 kDa |
| | transporting F1 complex | | | |
| 16. | Laminin-binding protein | Up-regulated | P08865 | 32.8 kDa |
| 17. | Minichromosome maintenance | Up-regulated | Q53FG5 | 82.2 kDa |
| | deficient protein 5 variant | | | |
| 18. | Keratin 9 | Up-regulated | P35527 | 61.2 kDa |
| 19. | Keratin 10 | Up-regulated | P13645 | 59.5 kDa |
| 20. | Napsin A aspartic peptidase | Up-regulated | Q8WWD9 | 45.4 kDa |
| 21. | M2-type pyruvate kinase | Up-regulated | P14618 | 57.9 kDa |
| 22. | Apolipoprotein A-I | Down-regulated | P02647 | 30.8 kDa |

A list showing the biomarkers in FIGS. 1A and 1B and the change in the spot intensity of the biomarkers in FIGS. 2 to 20 are summarized in Table 2 below.

TABLE 2
Summary of the change in the spot intensity of biomarkers in 3 NSCLC
patients

| | | Ratio of | Ratio | Ratio |
|---|---|---|---|---|
| | | of differential | of differential | differential |
| Spot | | expression | expression | expression |
| Number | Protein Identity | in col. A | in col. B | in col. C |
| 1A | Peroxiredoxin II | −4.55 | −11.11 | −4.92 |
| 1B | Peroxiredoxin II | −5.91 | −10.34 | −3.54 |
| 2 | Chaperonin | 2.46 | 10.57 | 17.14 |
| 3 | Serum amyloid P component | −6.39 | −7.38 | −17.15 |
| 4 | Annexin V | 2.00 | 1.51 | 4.58 |
| 5 | Dihydropyrimidinase-like 2 | −4.10 | −2.07 | −14.37 |
| | protein | | | |
| 6 | Glutamate carboxypeptidase | 2.69 | 2.47 | 7.10 |
| 7 | 2,3-bisphosphoglycerate | −2.39 | −3.77 | −2.42 |
| | mutase | | | |
| 8 | Thymidine phosphorylase | 7.69 | 3.57 | 2.68 |
| 9 | Prolyl-4 hydroxylase | 7.27 | 2.39 | 13.05 |
| 10 | Selenium binding protein 1 | −7.54 | −6.03 | −3.67 |
| 11 | β-mitochondrial ATP synthase | 9.35 | 10.96 | 10.83 |
| | H⁺ transporting F1 complex | | | |
| 12 | Laminin-binding protein | 7.14 | 3.74 | 2.66 |
| 13 | Minichromosome | 2.10 | 2.00 | 2.04 |
| | maintenance deficient protein | | | |
| | 5 variant | | | |
| 14 | Keratin 9 | 4.68 | 5.04 | 4.08 |
| 15 | Keratin 10 | 2.58 | 5.99 | 1.83 |
| 16 | Napsin A aspartic peptidase | 2.33 | 8.37 | 10.35 |
| 17 | M2-type pyruvate kinase | 23.00 | 6.47 | 5.38 |
| 18 | Apolipoprotein A-I | −7.14 | −13.93 | −9.30 |

The ratio in Table 2 and FIGS. 2 to 20 may be calculated as:

Ratio=(optical density of the protein spot*spot area*spot volume in tumor tissue)/(optical density of the same protein spot*spot area*spot volume in non-tumor tissue)

2. Panel of Biomarkers

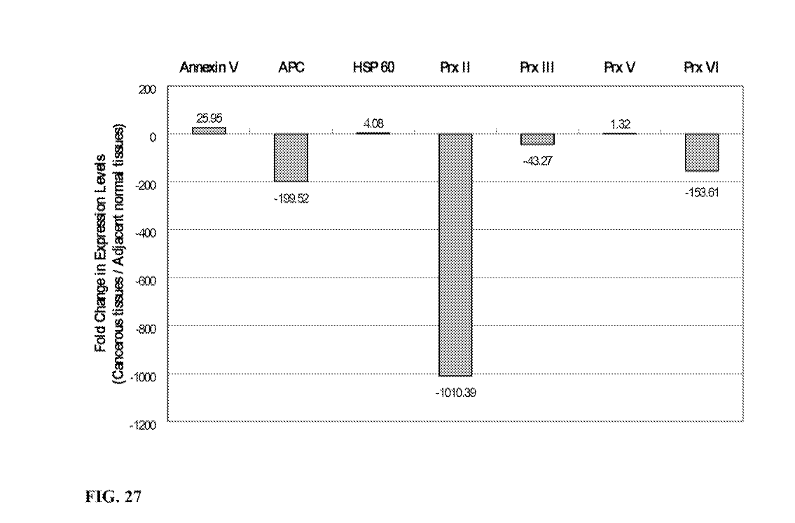

There is also provided using a panel of at least two biomarkers, as selected from Table 1, for early detection of lung cancer, prognosis of cancer treatment, and design of treatment protocols using these proteins. For example, the differential expression of a panel of six biomarkers that included annexin V, amyloid P component, chaperonin (Hsp 60), Prx II, Prx III, Prx VI are depicted in FIG. 27 and described in Example 7.

A method for identifying, diagnosing, and monitoring cancerous tissues may include measuring the expression of a panel of biomarkers in a biological sample, and comparing the expressions of the panel against a panel of normal values. When a differential expression of each biomarker in the lung tissue sample is more than 1.5-fold of the normal values, the lung tissue sample is cancerous. The panel of biomarkers may include peroxiredoxin II, peroxiredoxin III, peroxiredoxin VI, chaperonin, amyloid-P-component, and annexin V. The biological sample may include lung tissue, lung tissue biopsy and/or serum.

3. Sample Preparation of Tumor and Non-Tumorous Tissues

Lung tumor tissue and adjacent paired non-tumorous tissue may be collected from patients undergoing surgery. After histopathological analysis of the specimens, leftovers may be kept as archives. One pair of adjacent normal (Sample N) and tumor tissues (Sample T) from the same patient may be included in each set of samples. Protein extracts may be prepared by homogenizing the entire tissue with phosphate buffer saline containing a protease inhibitors cocktail (available from Sigma, U.S.A.). After homogenization, the suspension may be centrifuged at 16000 g for 15 min at 4° C., and the supernatant may be isolated. Protein concentrations may be determined by using Bradford protein assay (available from Bio-Rad Laboratories Ltd in Hercules, Calif., U.S.A.). Protein samples may be stored at −80° C. until use. Since the lung tissue samples may have various degrees of haemolysis, equal protein loading corresponding to lung tissues may be normalized by relative expression of housekeeping protein-β-actin prior to 2D gel electrophoresis.

3.1 2-D Gel Electrophoresis and Image Analysis

A. Isoelectric Focusing

2-D gel electrophoresis may be performed on PROTEAN IEF cell and PROTEAN II XL system (available from Bio-Rad Laboratories Ltd in Hercules, Calif., U.S.A.). In the first dimension isoelectric focusing (IEF), the proteins may be resolved on pre-cast 18 cm linear pH gradient 4-7 IPG strips (available from Bio-Rad Laboratories Ltd). Specifically, the protein samples may be added into the rehydration buffer (2 M thiourea, 7 M urea, 4% (v/v) CHAPS, 10% (v/v) isopropanol, 5% (v/v) glycerol, 64 mM DTT and 1% IPG buffer) and rehydrated actively onto the IPG strips. After rehydration, the subsequent IEF may be performed at 20° C. with a PROTEAN IEF Cell (available from Bio-Rad Laboratories Ltd). Voltage may be applied as follows: 1 hour at 500 V, 1 hour at 1000 V, 2 hour at 4000 V, 4 hours at 8000 V and 80 kVhr at 8000V, resulting in a total voltage of 100 kVhr.

B. Equilibration of IPG Strip

Following IEF, the IPG strips may be equilibrated with 1% (w/v) DTT in the equilibration buffer (50 mM Tris-HCl (pH 8.8), 6 M urea, 39% (v/v) glycerol, 2% (w/v) SDS and 0.006% (w/v) bromophenol blue) for 15 minutes and subsequently in a solution of 1% (w/v) iodoacetamide in the same equilibration buffer for 15 minutes.

C. SDS-PAGE

After IPG strip equilibration, the IPG strips may be rinsed with ddH₂O and then may be placed on top of 10% SDS-PAGE and sealed with 0.5% agarose in electrophoresis buffer for the second dimension. Electrophoresis may be performed for the first 30 minutes at 15 mA/gel followed by 30 mA/gel until the bromophenol blue dye front may reach the lower end of the gel, using a PROTEAN II xi 2D cell electrophoresis unit (available from Bio-Rad Laboratories Ltd). Proteins may be visualized by mass spectrometry compatible silver staining for image analysis.

D. Compatible Silver Staining

After SDS-PAGE, the gels may be fixed for 1 hour initially in fixation solution containing 40% (v/v) ethanol and 10% (v/v) acetic acid. The gels may then be sensitized in a solution containing 30% (v/v) ethanol, 0.2% (w/v) sodium thiosulphate, 6.8% (w/v) sodium acetate and 0.125% (v/v) glutaraldehyde for 30 minutes, and then may be washed with distilled water (for example, 3 times for 5 minutes each).

Subsequently the gels may be stained for 20 minutes in 0.25% (w/v) silver nitrate with 0.015% (v/v) formaldehyde before washing with distilled water again (for example, 2 times for 1 minute each). Protein spots in gels may then be developed in 2.5% (w/v) sodium carbonate containing 0.0074% (v/v) formaldehyde. The development may be stopped using 1.5% (w/v) EDTA.

E. Image Analysis

Silver-stained gels may be scanned at 300 dots per inch resolution using a conventional scanner (available from Epson Perfection 1200U). All spot detection and spot comparisons may be made using Melanie 3.08 (available from Gene-Bio in Switzerland). Spot intensity may be normalized by plotting the optical density of each paired and matched protein spots in two comparing gels in a scatter plot. The differential expression may be confirmed only when it was consistently present in all three gels. Moreover, the significance may be regarded only when the difference in spot optical density is at least by 1.5-fold.

3.2 Protein Identification by MALDI-TOF Mass Spectrometry

A. Protein Preparation

Protein spots showing a differential expression may be manually excised, destained, reduced with 10 mM dithiothreitol (DTT), and alkylated with 55 mM iodoacetamide. Enzymatic digestion may be performed overnight with sequencing grade-modified trypsin (available from Promega in Madison, Wis., USA) at 37° C. The digested peptides may be extracted with 50% (v/v) acetonitrile and 1% (v/v) trifluoroacetic acid. Ultra-sonication may be applied to facilitate the diffusion of peptides. All PMF and tandem MS spectra may be obtained using a Bruker AUTOFLEX III Smartbeam (available from Bruker Daltonics in Germany). MS/MS or PMF data may then be searched using Mascot software (available from MatrixScience in the U.K.) against NCBI database.

B. MALDI-TOF Analysis

The MALDI-TOF analysis may be performed with a Bruker AUTOFLEX III Smartbeam (available from Bruker Daltonic in Billerica, Mass., U.S.A.) instrument operated in the reflectron mode over the m/z range of 1000 to 3000 Da. For the analysis, 1 μl sample aliquot may be spotted onto a sample plate, which may be pre-spotted with 1 μl of matrix solution (2 mg/ml a-cyano-4-hydroxycinnamic acid, saturated in acetonitrile:0.1% TFA (1:1)) and allowed to air-dry. After drying, the sample spot may be washed with 1 μl of a 1% (v/v) TFA solution for approximately 30 sec. The TFA droplet may be gently blown off the sample spot with compressed air. The resulting diffused sample spot may be recrystallized using 1 μl of a solution of ethanol:acetone:0.1% (v/v) TFA (6:3:1 ratio). Reported spectra may include the summation of 200 laser shots. Spectra may be calibrated using Mass Calibration Kit (available from Bruker Daltonics) with an external calibration standard. The peptide masses generated via MALDI-TOF may be used to search the NCBI non-redundant (NCBInr) database using the Mascot search engine, and the Mowse score cut-off point for a positive identification may be 64.

C. Western Blotting

Protein samples may be resolved on one-dimensional SDS-PAGE and electro-blotted onto a nitro-cellulose (NC) membrane. After electro-blotting, the NC membrane may be blocked with 5% (w/v) skim milk in Tris buffer saline with 0.05% (v/v) Tween 20 (TBST) for 2 hours at room temperature. Subsequently, the membrane may be incubated with antibodies specifically against individual protein biomarker for 2 hours at room temperature. After six washings, the membrane may then be incubated with horse-radish peroxidase-conjugated antibodies for 1 hour at room temperature. Finally, the signal may be visualized with Super Signal ECL Kit (available from Promega in the U.S.A.). The intensities of the corresponding immunoreactive bands may measure as the black light units (BLU). The relative protein quantitation may be normalized against housekeeping protein, β-actin, which may act as internal standard.

Expression analysis may also be carried out by measuring the expression of a fragment of the biomarker protein. For example, an antibody specific for the unique fragment of the biomarker protein may be used to detect and quantify expression in the same way as the antibody could be used to detect the intact protein. Expression analysis may also be carried out by measuring the expression of mRNA encoding the biomarker protein, and/or expression of antibodies specifically against the intact protein and/or a fragment of biomarker protein.

The method described above may be used to detect the presence of early stages of NSCLC for screening, diagnostic and prognostic purposes. The method may be further developed for use in the determination of the efficacy of treatment on lung cancer due to surgery, radiation and chemotherapy, as well as further the design of targeted drug for cancer treatment.

4. Method of Use of Biomarkers

The protein biomarkers may be detected quantitatively and semi-quantitatively by different antibody-based techniques, different types of mass spectrometry (SELDI MS, MALDI TOF MS and tandem MS), or other means in biological samples (biopsy tissues, tumor tissues and blood fluids). Antibody-based techniques may be included different types of immunoassays, conventional or tissue microarray immunohistochemical analysis. Examples of immunoassays include sandwich immunoassays (ELISA or fluorescence-based immunoassays), nephelometry, and biosensors SELDI-based immunoassays.

4.1 Detection by Antibody-Based Techniques

The presence of protein biomarkers and their expression levels in the biological samples may be detected with immunohistochemical methods, or measured quantitatively by different types of immunoassays. Both of which are antibody-based techniques that may require a capture reagent, such as bio-specific antibodies, to specifically interact with the entire biomarker or a polypeptide of the biomarker (refer as antigen).

Antibodies may be monoclonal or polyclonal, and may refer generally to any immunologic capture agent such as IgG, IgM, IgA, IgD and IgE. Monoclonal antibodies may be used to bind to one site of a particular molecule, and therefore may provide a more specific and accurate result. Antibodies may be produced by immunizing animals or poultry with the entire antigen (protein biomarker) or a specific polypeptide of the antigen. Examples of immunizing animals include rats, mice, and rabbits. Examples of poultry include hens. The antigen used for immunization may be isolated from the samples or synthesized by recombinant protein technology.

Primary antibody may provide the specific recognition for detecting or quantifying the protein biomarker. Secondary detection system may utilize enzyme (e.g. enzyme immunoassay (EIA)), radioisotopes (e.g. 1-125 radioimmunoassay (RIA)), magnetic labels (e.g. magnetic immunoassay (MIA)), fluorescence, or other conjugated secondary antibodies.

With the use of specific antibodies, an immunohistological staining of tissue section for pathological examination may be obtained from conventional and/or tissue microarray immunohistochemical analysis. Besides, tissues may also be extracted for the liberation of protein biomarkers for western blot or dot/slot assay. In this technique, which is based on the use of cationic solid phases, quantitation of protein biomarker may be accomplished by comparing isolated biomarker standards to a known concentration. A molar concentration of protein biomarker may aid to set the standard values of the biomarker content for different tissues, fecal matter or body fluids (e.g. serum, plasma, urine, synovial fluid, spinal fluid. Finally, the normal appearance of biomarker amounts may be set using values from healthy subjects, and can be compared against those obtained from a test subject. Other immuno-techniques may include agglutination, nephelometry, turbidimetry and biosensors, and SELDI-based mass spectrometry.

4.2 Detection by Mass Spectrometry

In addition to antibody-based techniques, the protein biomarkers may also be detected and quantified by mass spectrometry. Mass spectrometry is a method that employs a mass spectrometer to detect ionized protein markers or ionized peptides as digested from the protein markers by measuring the mass-to-charge ratio (m/z). For example, analytes may be introduced into a sample inlet and ionized in an ionization source, and the ionized analytes may be introduced into mass analyzers for mass separation before their mass being measured by a detector.

Differences in the sample inlet, ion source, and mass analyzer may generally define the type of instrument and its capacities. For instance, a sample inlet may include a capillary-column liquid chromatography source, or may include a direct probe or a stage as used in matrix-assisted laser desorption. Examples of the ion source may include an electrospray, a nanospray, a microspray, and a matrix-assisted laser desorption. Example of the mass analyzers may include a quadrupole mass filter, an ion trap mass analyzer, and a time-of-flight mass analyzer. Preferably, matrix assisted laser deionization/ionization-time of flight (MALDI-TOF) mass spectrometry, tandem mass spectrometry, and surface enhanced laser desorption/ionization (SELDI) mass spectrometry may be used.

A mass spectrometric method for identifying a lung cancer biomarker or the corresponding peptide may include providing a biological sample from a subject having a lung cancer, concentrating digested peptides belonging to protein biomarkers in the biological sample, separating one or more of the digested peptides, and subjecting the separated peptides to mass spectrometry to identify at least one of the separated peptides. One or both of the presence and amount of the at least one identified peptide may indicate that the peptide is a lung cancer biomarker peptide. For example, the amount of at least two biomarkers in the biological sample from a subject may be measured using mass spectrometry. One or both of an increase in the amount of signal at the m/z ratio values specific to the digested peptides from an up-regulated protein biomarker, and a decrease in the amount of signal at the m/z ratio values specific to the digested peptides from a down-regulated protein biomarker in the biological sample as compared to a non-lung cancer reference amount of the signals may indicate that the subject may have lung cancer. SELDI is a technique based on the pretreatment of a biological fluid or tissue extract with various proteomic chips, performing protein extractions based on hydrophobic, ion-exchange, metal binding, or other interactions. The bound proteins may then be subjected to mass spectrometric analysis.

5. Detection of Biomarkers in Blood Serum

Biomarkers may be found in blood serum, as suggested by the two examples below.

Prostate specific antigen (PSA) is a biomarker used commonly for diagnosis and management of prostate cancer. PSA is an androgen-regulated, serine protease produced by both normal prostate epithelial and prostate cancer cells. A significant up-regulation of PSA may be observed in prostate cancer tissues, ranging from 0.4-140 ρg. Its elevated level may be detected in cancer patient serum. In clinical applications, testing for the elevated serum levels of PSA may be used as a screening method for prostate cancer.

An up-regulation of heat shock protein 70 (HSP70) has been identified in hepatocellular carcinoma tissues, colorectal cancer tissues, and cervical cancer tissues. With the use of proteomic approach, an elevation in the sera of autoantibodies reacting with hsp 70 and an increase of hsp 70 in serum may be observed in cancer patients. Since autoantibodies may be produced in cancer patients from an early stage, and the amount of autoantibodies against hsp70 and the level of hsp 70 may be detectable in patient's serum, HSP70 may be a suitable biomarker for cancer screening in blood.

Having described embodiments of the biomarkers and method with reference to the accompanying drawings, it is to be understood that the biomarkers and method are not limited to the precise embodiments, and that various changes and modifications may be effected therein by one having ordinary skill in the art without departing from the scope or spirit as defined in the appended claims.

Furthermore, it should be understood that the biomarkers and method are not limited to the precise embodiments described below and that various changes and modifications thereof may be effected by one skilled in the art without departing from the spirit or scope of the invention. For example, elements and/or features of different illustrative embodiments may be combined with each other and/or substituted for each other within the scope of this disclosure and appended claims.

### EXAMPLES

### Examples 1

### Expression of Amyloid P Component in NSCLC Tissues and its Adjacent Non-Tumor Tissues

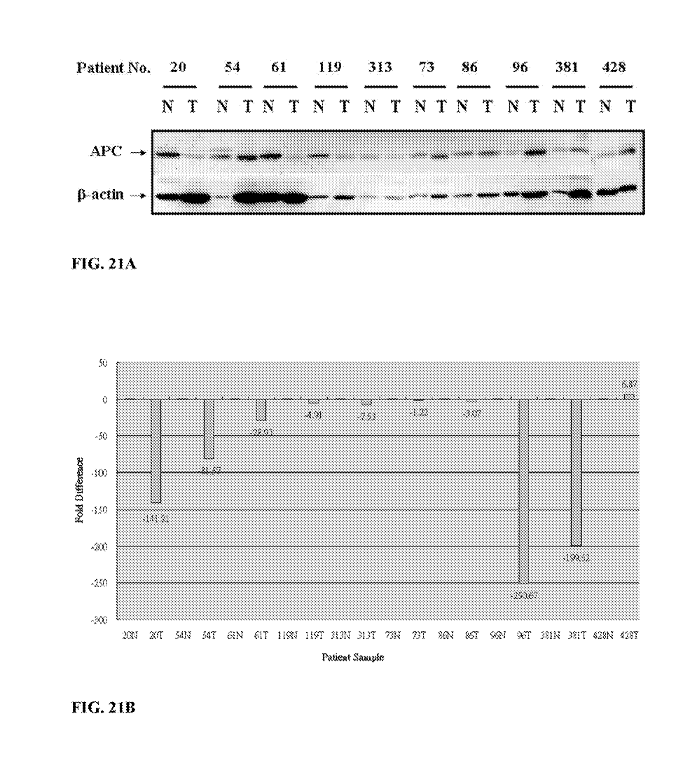

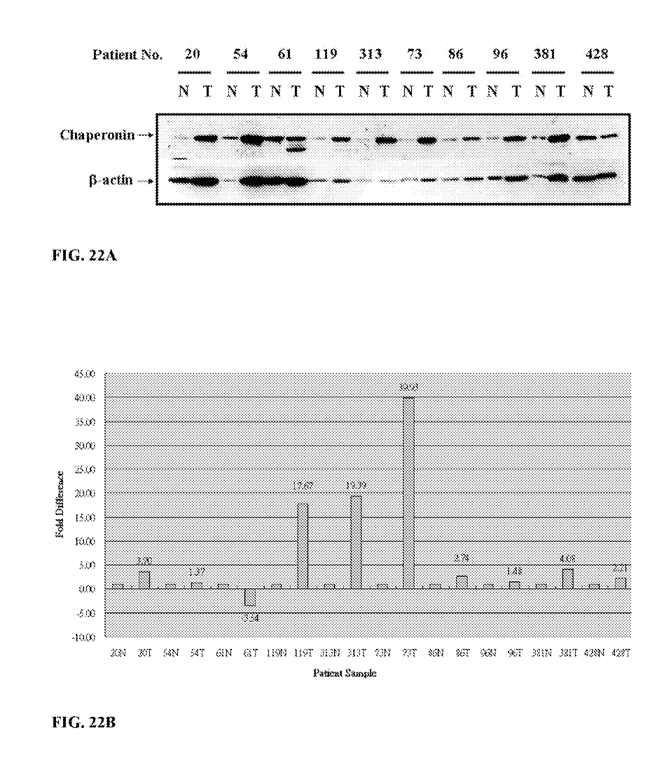

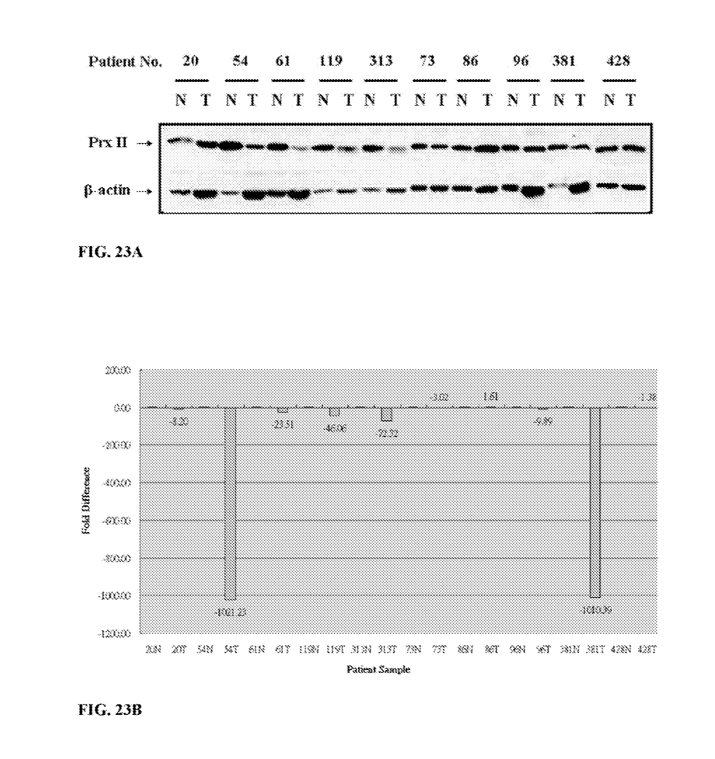

An immunoreactive band for amyloid P component (APC) with an apparent molecular weight of about 25 kDa was specifically detected in tumor (T) and non-tumor tissues (N), as shown in FIG. 21A. The band intensities were quantified by their black light units (BLU). The relative expression level of amyloid P component was normalized equally by the internal control, β-actin in either tumor tissues or the paired non-tumor tissues prior to comparison. This was because the lung tissue samples had various degrees of haemolysis, and the amounts of stable expressed tissue protein, β-actin, may vary greatly in the analyzed tissue samples.

After normalizing with β-actin, the relative expression level of amyloid P component was reduced with more than 1.5-fold in eight of out ten tumors analyzed when compared to the adjacent non-tumor tissues, as depicted in FIG. 21B. Only one of the samples, patient no. 428, showed an increased expression of APC in tumor tissues.

The data has therefore shown that APC may be used as a biomarker for identifying, diagnosing, and monitoring cancerous lung tissues.

### Example 2

### Expression of Chaperonin in NSCLC Tissues and its Adjacent Non-Tumor Tissues

An immunoreactive band for chaperonin with an apparent molecular weight of about 60 kDa was specifically detected in tumor (T) and non-tumor tissues (N), as shown in FIG. 22A. The relative expression level of chaperonin was normalized equally by the internal control, β-actin in either tumor tissues or the paired non-tumor tissues prior to comparison.

The relative expression level of chaperonin was increased with more than 1.5-fold in seven out of ten tumors analyzed when compared to the adjacent non-tumor tissues, as depicted in FIG. 22B. A decreased expression was only found in one of the samples, patient no. 61. No significant changes were observed in patient nos. 54 and 96.

The data has therefore shown that chaperonin may be used as a biomarker for identifying, diagnosing, and monitoring cancerous lung tissues.

### Example 3

### Expression of Peroxiredoxin II in NSCLC Tissues and its Adjacent Non-Tumor Tissues

An immunoreactive band for peroxiredoxin II with an apparent molecular weight of about 25 kDa was specifically detected in tumor (T) and non-tumor tissues (N), as shown in FIG. 23A. The relative expression level of peroxiredoxin II was normalized equally by the internal control, β-actin in either tumor tissues or the paired non-tumor tissues prior to comparison.

The relative expression level of peroxiredoxin II was decreased with more than 1.5-fold in eight out of ten tumors analyzed when compared to the adjacent non-tumor tissues, as depicted in FIG. 23B. No significant changes were observed in patient nos. 86 and 428.

The data has therefore shown that peroxiredoxin II may be used as a biomarker for identifying, diagnosing, and monitoring cancerous lung tissues.

### Example 4

### Expression of Peroxiredoxin III in NSCLC Tissues and its Adjacent Non-Tumor Tissues

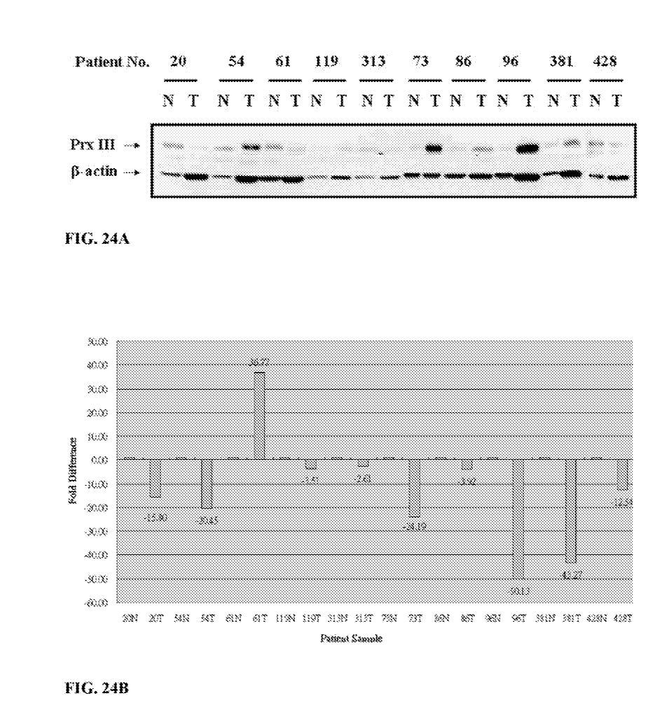

An immunoreactive band for peroxiredoxin III with an apparent molecular weight of about 25 kDa was specifically detected in tumor (T) and non-tumor tissues (N), as shown in FIG. 24A. The relative expression level of peroxiredoxin III was normalized equally by the internal control, β-actin in either tumor tissues or the paired non-tumor tissues prior to comparison.

The relative expression level of Peroxiredoxin III was decreased with more than 1.5-fold in nine out of ten tumors analyzed when compared to the adjacent non-tumor tissues, as depicted in FIG. 24B. Only one of the samples, patient no. 61, showed an increased expression of Peroxiredoxin III in tumor tissues.

The data has therefore shown that peroxiredoxin III may be used as a biomarker for identifying, diagnosing, and monitoring cancerous lung tissues.

### Example 5

### Expression of Peroxiredoxin VI in NSCLC Tissues and its Adjacent Non-Tumor Tissues

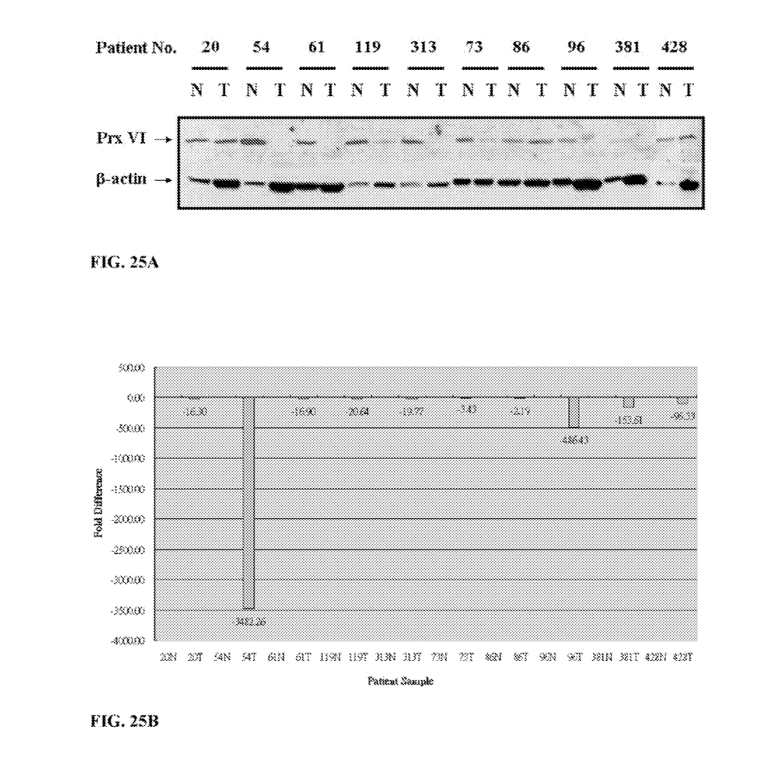

An immunoreactive band for peroxiredoxin VI with an apparent molecular weight of about 25 kDa was specifically detected in tumor (T) and non-tumor tissues (N), as shown in FIG. 25A. The relative expression level of peroxiredoxin VI was normalized equally by the internal control, β-actin in either tumor tissues or the paired non-tumor tissues prior to comparison.

The relative expression level of peroxiredoxin VI was decreased with more than 1.5-fold in all of the tumors analyzed when compared to the adjacent non-tumor tissues, as depicted in FIG. 25B. The data has therefore shown that peroxiredoxin VI may be used as a biomarker for identifying, diagnosing, and monitoring cancerous lung tissues.

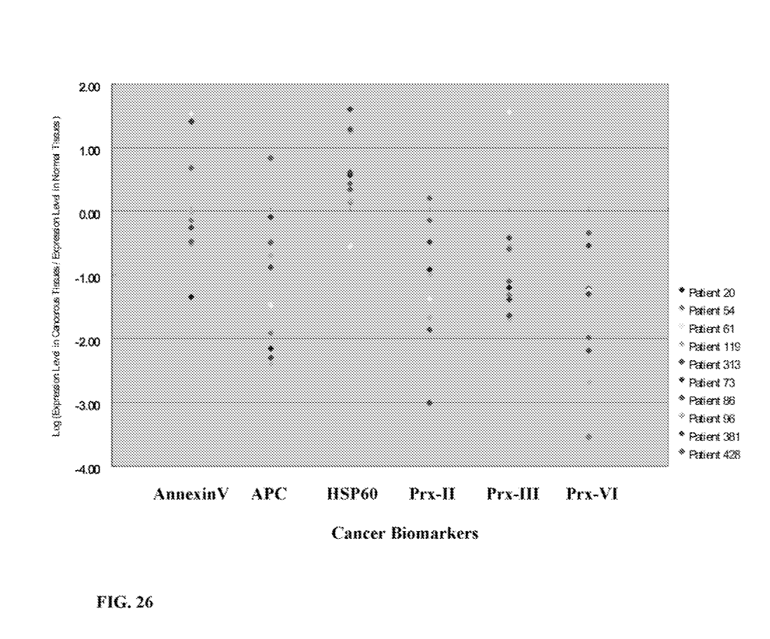

A dot chart showing the log value of fold change in the expression of amyloid P component, chaperonin, peroxiredoxin II, III and VI in tumor tissues from NSCLC patients is depicted in FIG. 26.

### Example 6

### Biomarker Panel

A panel of six biomarkers was created that included annexin V, APC, chaperonin (Hsp 60), Prx II, Prx III and Prx VI. The relative expression level of each of the six biomarkers was measured and normalized in the same way as described in Examples 1 to 5. Five out of the six biomarkers exhibited more than a 1.5-fold in change according to its corresponding expression level, as depicted in FIG. 27.

The data therefore has shown a panel of biomarkers may significantly increase the accuracy in identifying, diagnosing, and monitoring cancerous lung tissues.

While the biomarkers and method have been described, it should be understood that the biomarkers and method are not so limited, and modifications may be made. The scope of the biomarkers and method are defined by the appended claims, and all devices that come within the meaning of the claims, either literally or by equivalence, are intended to be embraced therein.

What is claimed is:

1. A method for identifying, diagnosing, and monitoring cancerous lung tissues in a subject, comprising:
measuring expression of at least one biomarker in a biological sample in said subject; and
comparing said expression against a normal value,
wherein, when a differential expression of said at least one biomarker between said biological sample and said normal value is more than 1.5-fold, said lung tissue is cancerous, and
wherein said at least one biomarker is selected from the group consisting of peroxiredoxin I, peroxiredoxin II, peroxiredoxin III, peroxiredoxin IV, peroxiredoxin VI, chaperonin, amyloid-P-component, annexin V, dihydropyrimidinase-like 2 protein, glutamate carboxypeptidase, 2,3-bisphosphoglycerate mutase, thymidine phosphorylase, prolyl-4 hydroxylase, selenium binding protein 1, β-mitochondrial ATP synthase H⁺ transporting F1 complex, laminin-binding protein, minichromosome maintenance deficient protein 5 variant, keratin 9, keratin 10, napsin A aspartic peptidase, M2-type pyruvate kinase, and apolipoprotein A-I.

2. The method of claim 1, wherein said biological sample comprises lung tissue biopsy.

3. The method of claim 1, wherein said biological sample comprises serum.

4. The method of claim 1, wherein said measuring expression comprises using an antibody-based technique to measure expression of biomarker protein.

5. The method of claim 4, wherein said antibody-based technique comprises an enzyme-linked immunosorbent assay.

6. The method of claim 4, wherein said antibody-based technique comprises an immunohistochemical analysis.

7. The method of claim 4, wherein said antibody-based technique comprises a tissue microarray immunohistochemical analysis.

8. The method of claim 1, wherein said measuring expression comprises using mass spectrometry to measure expression of biomarker protein.

9. The method of claim 8, wherein said mass spectrometry comprises surface enhanced laser desorption/ionization.

10. The method of claim 1, wherein said expression is over-expression, and said at least one biomarker is selected from the group consisting of chaperonin, annexin V, glutamate carboxypeptidase, thymidine phosphorylase, prolyl-4 hydroxylase, β-mitochondrial ATP synthase H⁺ transporting F1 complex, laminin-binding protein, minichromosome maintenance deficient protein 5 variant, keratin 9, keratin 10, napsin A aspartic peptidase and M2-type pyruvate kinase.

11. The method of claim 1, wherein said expression is under-expression, and said at least one biomarker is selected from the group consisting of peroxiredoxin I, peroxiredoxin II, peroxiredoxin III, peroxiredoxin IV, peroxiredoxin VI, amyloid-P-component, dihydropyrimidinase-like 2 protein, 2,3-bisphosphoglycerate mutase, selenium binding protein 1 and apolipoprotein A-I.

12. The method of claim 1, wherein said cancerous lung tissue comprises non-small cell lung carcinoma.

13. The method of claim 1, wherein said at least one biomarker comprises a panel of at least two biomarkers.

14. A method of determining the effectiveness of a treatment for a patient diagnosed with cancerous lung tissues, comprising:
measuring expression of at least one biomarker in a first biological sample from said patient before treatment;
measuring expression of said at least one biomarker in a second biological sample from said same patient taken during said treatment;
comparing said expression of said first sample before treatment against a normal value; and
comparing said expressions of said second sample after treatment against said normal value,
wherein, when a differential expression of said at least one biomarker between said first sample before treatment and said normal value is more than 1.5-fold than a differential expression of said at least one biomarker between said second sample after treatment and said normal value, said treatment is effective, and
wherein said at least one biomarker is selected from the group consisting of peroxiredoxin I, peroxiredoxin II, peroxiredoxin III, peroxiredoxin IV, peroxiredoxin VI, chaperonin, amyloid-P-component, annexin V, dihydropyrimidinase-like 2 protein, glutamate carboxypeptidase, 2,3-bisphosphoglycerate mutase, thymidine phosphorylase, prolyl-4 hydroxylase, selenium binding protein 1, β-mitochondrial ATP synthase H⁺ transporting F1 complex, laminin-binding protein, minichromosome maintenance deficient protein 5 variant, keratin 9, keratin 10, napsin A aspartic peptidase, M2-type pyruvate kinase, and apolipoprotein A-I.

15. The method of claim 14, wherein said biological sample comprises lung tissue biopsy.

16. The method of claim 14, wherein said biological sample comprises serum.

17. The method of claim 14, wherein said measuring expression comprises using an antibody-based technique to measure expression of biomarker protein.

18. The method of claim 14, wherein said measuring expression comprises using a mass spectrometry to measure expression of biomarker protein.

19. The method of claim 14, wherein said expression is over-expression, and said at least one biomarker is selected from the group consisting of chaperonin, annexin V, glutamate carboxypeptidase, thymidine phosphorylase, prolyl-4 hydroxylase, β-mitochondrial ATP synthase H⁺ transporting F1 complex, laminin-binding protein, minichromosome maintenance deficient protein 5 variant, keratin 9, keratin 10, napsin A aspartic peptidase and M2-type pyruvate kinase.

20. The method of claim 14, wherein said expression is under-expression, and said at least one biomarker is selected from the group consisting of peroxiredoxin I, peroxiredoxin II, peroxiredoxin III, peroxiredoxin IV, peroxiredoxin VI, amyloid-P-component, dihydropyrimidinase-like 2 protein, 2,3-bisphosphoglycerate mutase, selenium binding protein 1 and apolipoprotein A-I.

21. The method of claim 14, wherein said cancerous lung tissue comprises non-small cell lung carcinoma.

22. The method of claim 14, wherein said at least one biomarker comprises a panel of at least two biomarkers.
